(19) United States
(12) Patent Application Publication
(10) Pub. No.: US 2008/0086067 A1
(43) Pub. Date: Apr. 10, 2008

(54) Ear compression device

(76) Inventors: Duff M. Hay, Sandwich, IL (US); Benjamin D. Hay, Fithian, IL (US)

Correspondence Address: Duff M. Hay, 1509 Lake Holiday Drive, Sandwich, IL, 60548, US

(21) Appl. No.: 11/544,721

(22) Filed: Oct. 10, 2006

(51) Int. Cl. A61F 5/00

(52) U.S. Cl. 602/5

(57) ABSTRACT

An improved non-invasive ear compression dressing or splinting device is provided for prevention, treatment and recurrence of injuries to the outer ear such as auricular hematoma. The device includes a structure including a pair of first and second pads, and a pressure applying assembly. The pads are assembled in pairs in opposing, facing relation to compressibly engage the injured portion of an external ear. The pressure applying assembly includes a means for forcing one pad assembly towards the other in an infinitely adjustable, controlled manner creating a compression of the ear tissues required for proper healing between the two opposing pads.

### BACKGROUND OF THE INVENTION

Devices and surgical procedures to aid in the prevention and healing of injuries to the external ear are rudimentary, crude, and painful. The most common such injury is auricular hematoma typically inflicted during contact sports such as wrestling, boxing, or rugby which if left untreated, or improperly treated, results in the hematomas becoming fibrotic and disfiguring, a thickening of the tissue known as “cauliflower ear”. Many protective devices such as U.S. Pat. Nos. 5,504,945 and 5,615,417 are intended to protect the tissues of the ear although history indicates devices such as these to either be too cumbersome to use consistently, minimally effective, or too inconvenient.

In addition to use in the treatment of auricular hematomas, pressure dressings are also used in many other treatments of the ear, such as the covering and compressing of an acute wound to the ear, the excision of a skin cancer, the placement of a skin graft, the repair of a torn earlobe, the treatment of a localized burn, or the excision of a keloid which can form on the earlobe or other portions of the ear after piercing thereof. Similar injuries and treatments occur with ears of animals.

Auricular hematomas are caused by a blunt trauma or shearing force to the external ear that disrupts the adherence of the perichondrium or skin of the ear to the underlying cartilage and the subsequent filling of the subperichondrial space with blood. Application of pressure when discomfort and preliminary trauma is incurred helps to prevent more serious damage. Once more serious damage has occurred, standard treatment involves needle aspiration of the hematoma or, better, incision and drainage, followed by compression of the injured area to prevent re-accumulation of fluid and to allow re-apposition of the perichondrium to the underlying cartilage; the application of pressure is crucial. Avoiding infection is another important measure to prevent further complications.

Several different compression techniques using pressure dressings have been employed to keep the skin in the necessary close contact with the cartilage during healing. The pressure dressings generally fall into the categories of suture compression dressings, mastoid dressings (i.e. dressings attached to the head by adhesive tape or other such methods), and molds (e.g. silicone) which are used with or without suturing or mastoid dressings.

Mastoid dressings are generally disfavored because of their bulkiness and tendency to come loose or be dislodged. Molds generally tend to be expensive and time consuming to apply. Since molds are very closely conforming, they do, however, tend to apply a more uniform pressure with force vectors that are directed orthogonally to a greater area of the structure involved in the injury. However, as the injured area becomes more or less inflamed either with the progression of healing and reduction of inflammation or by increased trauma and increase in inflammation, molds are unable to compensate for the adjustment in force required to maintain constant pressure.

Suture dressings, while invasive, are less bulky and more often effective. The most common method of applying pressure to the effected area involves suturing pressure dressing materials (often cotton balls or dental rolls with an antibiotic/antiseptic applied) positioned on opposing sides of the injured portion of the ear. The sutures are passed through the cartilage of the ear to gently squeeze the skin and cartilage together between the dressings. This method often does not provide evenly distributed pressure over the injured area and as a result, blood can re-accumulate under the skin to reform the hematoma. Reformation of the hematoma requires repeated aspiration of the accumulated blood. In order to provide a more evenly distributed pressure over the entire area of the injury, multiple sutures are necessary. Not only do these sutures through the ear cause much pain, but the risk of infection increases; with each aspiration or re-incision, infection potential is greater.

U.S. Pat. No. 5,827,212 attempts to more evenly distribute the pressure over the injured area although it still involves painful sutures through the ear tissue, introduces an increased risk of infection, and does not allow for adjustment of the applying force once installed.

U.S. Pat. No. 5,295,950 provides a non-invasive resolution of providing pressure to the outer ear utilizing a ductile metal strip with cushioning pads, eliminating the pain and risk of infection associated with penetrating the ear tissue with sutures. However, the force and resulting pressure applied to the ear is fixed by the physical nature of the ductile metal and amount of pressure applied when installed. No adjustability which is critical over time is included in this design other than removing and reapplying the device. Since the healing process takes several weeks for the skin to reattach, the dressing must be left in position for extended periods. As the healing process continues and the fluid increases or decreases, this method is unable to adjust for the change in thickness of the ear and pressure within the subperichondrial space. Loose dressings become less effective in maintaining sufficient pressure for complete healing and thickening and permanent deformity of the tissue can result.

A concept used for applying pressure on various other jointed body parts as in ankle, knee, neck or back splints and supports is a pneumatic concept and is utilized in U.S. Pat. Nos. 5,125,400, 5,316,547, 5,348,530, 5,407,421, 5,520,622, 5,542,911, and 5,623,723 although not specifically for ears.

It is an object of the present invention to provide a non-invasive device which provides a constant, evenly applied pressure over the effected area of the injured ear, by being able to be easily adjusted upon initial application and over the course of the healing process of several weeks.

It is another object of the present invention to provide a device which reduces the pain associated with treatments currently used.

A third object of the present invention is to minimize the risk of infection associated with treatments commonly in use.

It is a fourth object of the present invention to provide a reusable device.

The final object of the present invention is to provide a device that can adjust infinitely the pressure applied.

### SUMMARY OF THE INVENTION

Accordingly, the principal object of this invention is to provide a non-invasive compression type device for aiding in the prevention, treatment and healing of injuries of the external ear (pinna). It provides an infinitely adjustable and re-useable device for continuously applying even pressure directly to the outer ear often needed in the treatment of auricular hematoma.

A structure which extends over the cartilaginous rim portions of the ear or structures on either side of the external ear that allows application of pressure to the effected area is employed which incorporates pads or cushions. Application of pressure is accomplished by adjustably moving one of the pads closer towards the other creating a progressively smaller space between the pads within which the skin and tissue of the ear are caused to compress and re-adhere or remain intact; any fluids within the spaces of the perichondrium are forced out and can not re-enter.

Methods for creating the force for applying pressure are numerous and include mechanisms such as a screw, eccentric cam, inclined plane, fluidic, electromagnetic, and magnetic. These methods can provide infinite adjustability, allows re-use of the device if removed from the ear for any reason, and manually compensates for increases or reductions in pressure of the subperichondrial space in the effected area from increasing inflammation or decreasing amounts of fluid due to healing or other factors.

### VIEWS OF THE DRAWINGS

In order that all of the structural features for attaining the objects of this invention may be readily understood, reference is made to the accompanying drawings in which:

FIG. 1 is a side view showing the application of the present invention to an outer ear;

FIG. 2 is a rear view of the structure of FIG. 1 with one example of a screw thread pressure applying mechanism;

FIG. 3 is a rear view of the structure of FIG. 1, with the pressure applying mechanism located on the opposite (inside, close to the head) side of the ear as that shown in FIG. 2;

FIG. 4 is a section view of the structure of FIG. 2 taken along line 4-4 of FIG. 1 with one example of a screw thread pressure applying mechanism;

FIG. 5 is a section view similar to FIG. 4 with a second example of a screw thread pressure applying mechanism;

FIG. 6 is a section view similar to FIG. 4 with a third example of a screw thread pressure applying mechanism;

FIG. 7 is a section view similar to FIG. 4 with a fourth example of a screw thread pressure applying mechanism;

FIG. 8 is a section view similar to FIG. 4 with an example of an eccentric cam pressure applying mechanism;

FIG. 9 is a section view similar to FIG. 4 with an example of an inclined plane pressure applying mechanism;

FIG. 10 is a section view similar to FIG. 4 with an example of a fluidic (pneumatic) pressure applying mechanism;

FIG. 11 is a section view similar to FIG. 4 with an example of an electro-magnetic solenoid pressure applying mechanism;

FIG. 12 is a section view similar to FIG. 4 with an example of a magnetic pressure applying mechanism.

### DETAILED DESCRIPTION OF THE DRAWINGS

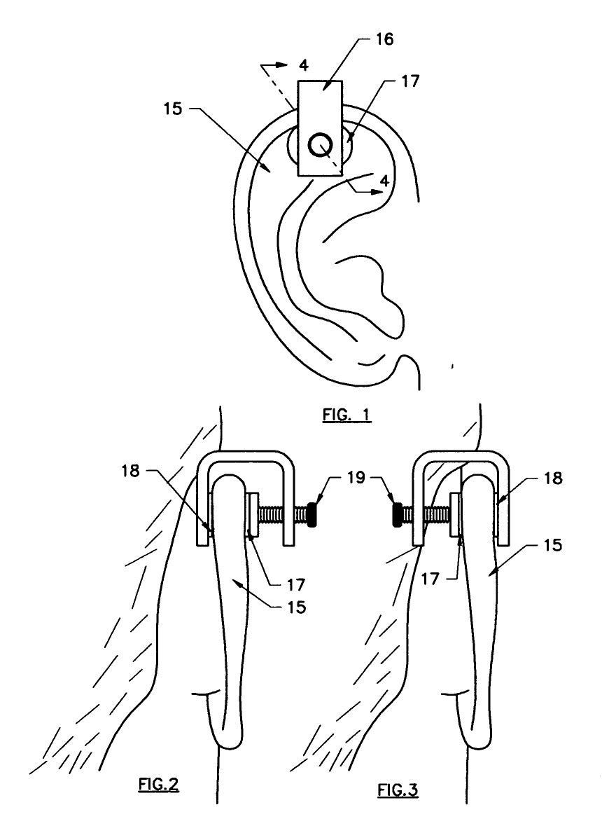

FIG. 1 shows a side view of the pinna 15 or outer ear with a structure 16 of the present invention utilizing one example of a screw thread pressure applying mechanism. Pad 17 is shown on the outside surface of the pinna 15 in relative position depending on specific location of the hematoma and to assure adequate compression of the underlying tissues of the perichondrium.

FIG. 2 is a rear view of FIG. 1 with the screw thread pressure applying device located on the outer (away from the head) side of the pinna 15. As the screw thread 19 is adjusted so as to reduce the space between pads 17 and 18, the underlying tissues of the perichondrium are compressed between said pads.

FIG. 3 is a rear view of FIG. 1 with the screw thread pressure applying mechanism located on the inner (close to the head) side of the pinna 15. As the screw thread 19 is adjusted so as to reduce the space between pads 17 and 18, the underlying tissues of the perichondrium are compressed between said pads. The operation of the pressure applying device is acting in the same manner as in FIG. 2 only from this alternate position.

FIG. 4 is a section view of the screw thread pressure applying device of FIG. 2 showing the attachment detail of the threaded screw 19 to the actuating pad 17 holder 20. The rotational movement of the threaded screw 19 is free with respect to the pad 17 holder 20 as the end of the threaded screw 19 is captured loosely within a cavity 21 in pad 17 holder 20 such that the pad 17 holder 20 is pushed or pulled as moves the threaded screw 19, thus allowing the pad 17 to maintain its fixed position on the pinna 15 while the inward or outward movement of the threaded screw relative to the structure 16 creates the force which is transmitted directly to the pad 17. This force creates an evenly applied pressure across the face of said pad 17 to the underlying tissues of the perichondrium and is equally resisted on the opposite side of the pinna 15 by the pad 18.

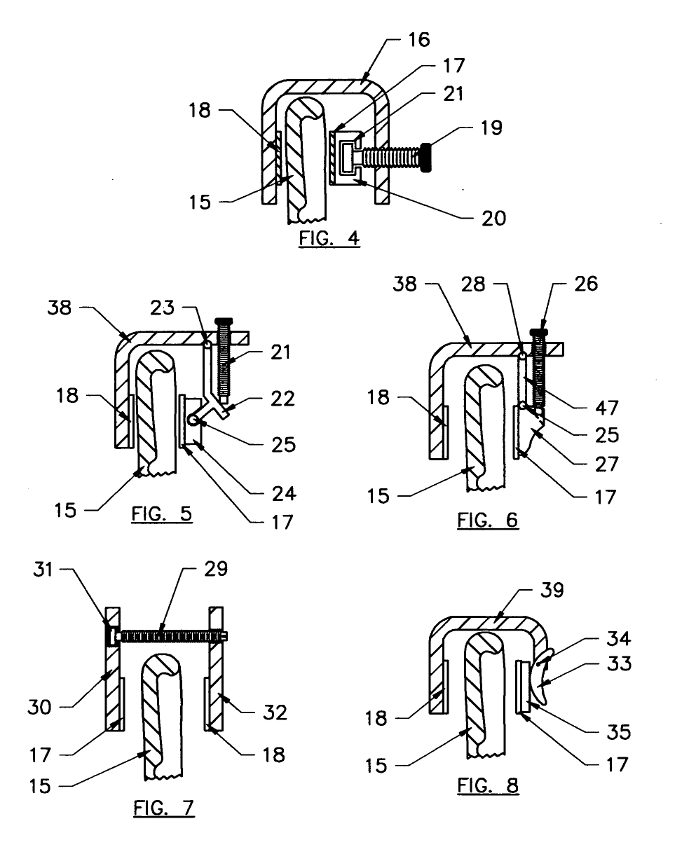

FIG. 5 is a section view similar to FIG. 4 of a structure 38 utilizing a second example of a threaded screw pressure applying device. As the rotational movement of the threaded screw 21 moves vertically downward, contact is made with the connecting arm 22 hinged at 23. The force is transmitted through this connecting arm 22 to the actuating pad 17 holder 24 via the pivoting joint 25 whereby the pad 17 is free to maintain its fixed position against the pinna 15. This creates an evenly applied pressure across the face of said pad 17 to the underlying tissues of the perichondrium which is equally resisted on the opposite side of the pinna 15 by the pad 18 via the structure 38. As noted in FIG. 3, the pressure applying device may also be located on the inner side of the pinna 15.

FIG. 6 is a section view similar to FIG. 4 of a structure 38 utilizing a third example of a threaded screw pressure applying device. As the rotational movement of the threaded screw 26 moves vertically downward, contact is made with the angled pad 17 holder 27 hinged at 28 with a connecting swing arm 47 hinged at 25. The force is transmitted through this angled pad 17 holder 27 to the actuating pad 17 such that the pad 17 is free to maintain its fixed position against the pinna 15 while the movement creates an evenly applied pressure across the face of said pad 17 to the underlying tissues of the perichondrium and is equally resisted on the opposite side of the pinna 15 by the pad 18 via the structure 38. As noted in FIG. 3, the pressure applying device may also be located on the inner side of the pinna 15.

FIG. 7 is a section view similar to FIG. 4 utilizing a fourth example of a threaded screw pressure applying device. The rotational movement of the threaded screw 29 is free with respect to the pad 17 holder 30 as the end of the threaded screw 29 is captured loosely within a cavity 31 in pad 17 holder 30 such that the pad 17 holder 30 is pushed or pulled as moves the threaded screw 29 in corresponding threads in pad 18 holder 32 thus allowing the pad 18 to maintain its fixed position on the pinna 15 while the inward or outward movement of the threaded screw 29 relative to pad 18 holder 32 creates the force which is transmitted directly to the pad 18. This force creates an evenly applied pressure across the face of said pad 18 to the underlying tissues of the perichondrium and is equally resisted on the opposite side of the pinna 15 by the pad 17. As noted in FIG. 3, the pressure applying device may also be located on the inner side of the pinna 15.

FIG. 8 is a section view similar to FIG. 4 of a structure 39 utilizing an eccentric cam lever 33 as the pressure applying device. As eccentric cam lever 33 rotates about pivot point 34, the increasing or decreasing radius at the point of contact with pad 17 holder 35 causes pad 17 holder 35 and pad 17 to move inward or outward relative to pinna 15. The force is transmitted through this pad 17 holder 35 to the pad 17 such that the pad 17 is free to maintain its fixed position against the pinna 15 while the movement creates an evenly applied pressure across the face of said pad 17 to the underlying tissues of the perichondrium and is equally resisted on the opposite side of the pinna 15 by the pad 18 via the structure 39. As noted in FIG. 3, the pressure applying device may also be located on the inner side of the pinna 15.

FIG. 9 is a section view similar to FIG. 4 of a structure 40 utilizing an inclined plane 36 as the pressure applying device. As the inclined plane 36 is pushed vertically downward within slot 38 in structure 40 contact is made with the pad 17 holder 37 causing pad 17 holder 37 and pad 17 to move inward or outward relative to pinna 15. The force is transmitted through pad 17 holder 37 to the pad 17 such that pad 17 and pad 17 holder 37 is free to maintain its fixed position against the pinna 15 while the movement creates an evenly app lied pressure across the face of said pad 17 to the underlying tissues of the perichondrium and is equally resisted on the opposite side of the pinna 15 by the pad 18 via the structure 40. As noted in FIG. 3, the pressure applying device may also be located on the inner side of the pinna 15.

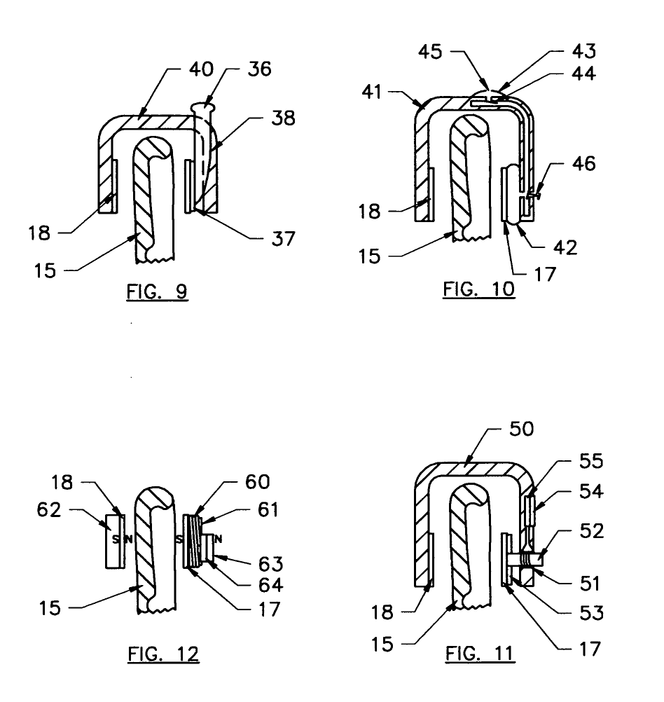

FIG. 10 is a section view similar to FIG. 4 of a structure 41 incorporating a fluidic pressure applying device. In this view, a pneumatic bladder 42 fills with air and expands thus causing pad 17 attached to pneumatic bladder 42 to move inward or outward relative to pinna 15. The force is transmitted through pad 17 such that the movement creates an evenly applied pressure across the face of said pad 17 to the underlying tissues of the perichondrium and is equally resisted on the opposite side of the pinna 15 by the pad 18 via the structure 41. As noted in FIG. 3, the pressure applying device may also be located on the inner side of the pinna 15.

Pressure is created by pressing on the flexible bulb 43 with the pressing member, usually a person's finger, covering the hole 45 with which air initially fills the space within the bulb. This pressing action and resulting collapse of the bulb causes the entrapped air to be pushed into the internal cavities of structure 41 through a pressure retention normally closed check valve 44 and into the pneumatic bladder 42. Pressure can be manually released from the internal cavities of structure 41 and pneumatic bladder 42 by pressing pressure retention normally closed valve 46.

FIG. 11 is a section view similar to FIG. 4 of a structure 50 incorporating an electro-magnetic solenoid pressure applying device. In this view, an electric solenoid coil 51 surrounds solenoid plunger 52 which is integrally attached to pad 17 holder 53. Electrically connected to one end of the electric solenoid coil 51 wire is an electrical circuit including a variable resistance capability, electricity flow direction control, and on/off switching capability 55 which connects to the positive side of disk battery 54. The other wire of the electric solenoid coil 51 is connected electrically to the negative side of disk battery 54. As electricity is applied to electric solenoid coil 51 from disk battery 54 the electro-magnetic field created causes the solenoid plunger 52 to move inward or outward relative to, pinna 15. The movement is transmitted through pad 17 such that the force creates an evenly applied pressure across the face of said pad 17 to the underlying tissues of the perichondrium and is equally resisted on the opposite side of the pinna 15 by the pad 18 via the structure 50. As noted in FIG. 3, the pressure applying device may also be located on the inner side of the pinna 15.

FIG. 12 is a section view similar to FIG. 4 incorporating a magnetic pressure applying device. In this view, an electric coil 60 surrounds permanent magnet 61 which is integrally attached to pad 17. Pad 18 is integrally attached to permanent magnet 62. Each permanent magnet 61 and 62 have north-south poles as indicated, and are oriented such that the opposing poles attract each other. The magnetic forces thus created cause an evenly applied pressure across the face of pad 17 to the underlying tissues of the perichondrium and is equally resisted on the opposite side of the pinna 15 by the pad 18.

Electrically connected to one end of the electric coil 61 wire is an electrical circuit including a variable resistance capability, electricity flow direction control, and on/off switching capability 64 which connects to the positive side of disk battery 63. The other wire of the electric coil 61 is connected electrically to the negative side of disk battery 63. As electricity is applied to electric coil 61 from disk battery 63 the electro-magnetic field, created causes the strength of permanent magnet to be reduced or increased depending on flow of electricity direction thereby allowing this electrical circuitry to provide adjustability of the magnetic attraction and resulting pressure transmitted across the face of pad 17 to the underlying tissues of the perichondrium which is equally resisted on the opposite side of the pinna 15 by the pad 18. As noted in FIG. 3, the pressure applying device may also be located on the inner side of the pinna 15.

In actual production designs, provisions for user friendliness, for economic manufacturing practices and techniques, and for maximizing effectiveness would be included and provided for which have not been identified as part of this patent but which would still render the device as being completely covered under the intent of this patent. The preferred embodiment previously described is illustrative of the principles of this invention. It should be understood, modifications can be made without departing from the scope of the invention.

What is claimed is:

1. A device comprising a structure which extends from the outer side of the external ear, around the cartilaginous rim portion of the ear, to the back side of the external ear adjacent to the outer side of the structure, with a pressure applying assembly as part of the structure to cause the space between the outer and back side of the structure to reduce thereby causing said structure to compress the ear tissue in this space.

2. The device of claim 1, wherein said structure utilizes a form fitting material referred to as pads that contact the tissues of the ear and transfer the forces created by the pressure applying assembly to evenly distribute pressure across the face of said pads. Said pads may contain a gelatinous fluid within internal cavities or may be of a consistent composition.

3. The device of claim 2, wherein said pressure applying assembly is able to be infinitely adjustable thereby being able to adjust the distance between adjacent opposing pads infinitely and, concurrently, adjust the pressure applied to the tissues of the ear infinitely.

4. The device of claim 3, wherein said pressure applying assembly utilizes a threaded screw mechanism.

5. The device of claim 3, wherein said pressure applying assembly utilizes an eccentric cam mechanism.

6. The device of claim 3, wherein said pressure applying assembly utilizes an inclined plane mechanism.

7. The device of claim 3, wherein said pressure applying assembly utilizes a fluidic mechanism.

8. The device of claim 3, wherein said pressure applying assembly utilizes an electro-magnetic solenoid mechanism.

9. A device comprising a structure on the outer side of the external ear and another structure on the back side of the external ear adjacent to the outer structure, with a magnetic pressure applying assembly as part of the structures to cause the space between the outer and back side structures to reduce thereby causing said structures to compress the ear tissue in this space.

10. The device of claim 9, wherein said structures utilize a form fitting material referred to as pads that contact the tissues of the ear and transfer the forces created by the magnetic pressure applying assembly to evenly distribute pressure across the face of said pads. Said pads may contain a gelatinous fluid within internal cavities or may be of a consistent composition.

11. The device of claim 10, wherein said magnetic pressure applying assembly is able to be infinitely adjustable thereby being able to adjust the distance between adjacent opposing pads infinitely and, concurrently, adjust the pressure applied to the tissues of the ear infinitely.

12. An ear compression dressing device comprising a structure which extends from the outer side of the external ear, around the cartilaginous rim portion of the ear, to the back side of the external ear adjacent to the outer side of the structure, with a pressure applying assembly as part of the structure to cause the space between the outer and back side of the structure to reduce thereby causing said structure to compress the ear tissue in this space.

13. The device of claim 12, wherein said structure utilizes a form fitting material referred to as pads that contact the tissues of the ear and transfer the forces created by the pressure applying assembly to evenly distribute pressure across the face of said pads. Said pads may contain a gelatinous fluid within internal cavities or may be of a consistent composition.

14. The device of claim 13, wherein said pressure applying assembly is able to be infinitely adjustable thereby being able to adjust the distance between adjacent opposing pads infinitely and, concurrently, adjust the pressure applied to the tissues of the ear infinitely.

15. The device of claim 14, wherein said pressure applying assembly utilizes a threaded screw mechanism.

16. The device of claim 14, wherein said pressure applying assembly utilizes an eccentric cam mechanism.

17. The device of claim 14, wherein said pressure applying assembly utilizes an inclined plane mechanism.

18. The device of claim 14, wherein said pressure applying assembly utilizes a fluidic mechanism.

19. The device of claim 14, wherein said pressure applying assembly utilizes an electro-magnetic solenoid mechanism.

20. An ear compression dressing device comprising a structure on the outer side of the external ear and another structure on the back side of the external ear adjacent to the outer structure, with a magnetic pressure applying assembly as part of the structures to cause the space between the outer and back side structures to reduce thereby causing said structures to compress the ear tissue in this space.

21. The device of claim 20, wherein said structures utilize a form fitting material referred to as pads that contact the tissues of the ear and transfer the forces created by the magnetic pressure applying assembly to evenly distribute pressure across the face of said pads. Said pads may contain a gelatinous fluid within internal cavities or may be of a consistent composition.

22. The device of claim 21, wherein said magnetic pressure applying assembly is able to be infinitely adjustable thereby being able to adjust the distance between adjacent opposing pads infinitely and, concurrently, adjust the pressure applied to the tissues of the ear infinitely.
